(19) United States
(12) Patent Application Publication
(10) Pub. No.: US 2010/0204595 A1
(43) Pub. Date: Aug. 12, 2010

(54) Heart monitoring system for treating a condition of a user

(76) Inventor: Elleen Marx, Murrieta, CA (US)

Correspondence Address: THE MATTHEWS FIRM, 2000 BERING DRIVE, SUITE 700, HOUSTON, TX, 77057, US

(21) Appl. No.: 12/378,246

(22) Filed: Feb. 12, 2009

(51) Int. Cl. A61B 5/0402; A61B 5/024

(52) U.S. Cl. 600/509; 600/508

(57) ABSTRACT

A system and method for facilitating treatment of a condition of a user, at least one symptom of the condition including an abnormal heart rate, is disclosed herein. A heart rate monitor measures one or more heart rate parameters of the user. A processor receives the parameters from the heart rate monitor and compares the parameters with threshold values in associated data storage and transmits a signal if the parameters exceed one or more of the threshold values. An audio device and a tactile device receive the signal from the processor. Responsive to the signal, the audio device provides an audible sound to the user, while the tactile device provides a tactile sensation to the user. The audible sound and tactile sensation are adapted to treat the user's condition, alleviate symptoms of the condition, or combinations thereof.

### FIELD

The embodiments herein relate generally to systems and methods for facilitating the treatment of condition of a user by detecting an abnormal heart rate caused by the condition and providing audible and/or tactile responses to the detected abnormal heart rate.

### BACKGROUND

In addition to heart patients, numerous other individuals suffer from a variety of conditions that can cause an abnormal heart rate. Individuals with neurological and/or behavioral disorders, such as anger management patients, anxiety sufferers, patients diagnosed with obsessive compulsive disorder, school children suffering from attention deficit disorder or one or more pervasive developmental disorders, persons with post traumatic stress disorder, and individuals with other similar conditions may exhibit a measurably abnormal heart rate. For such individuals, it is often possible to detect one or more symptoms of these conditions, as the symptoms occur, by monitoring one or more of the individual's heart rate parameters.

Numerous existing devices measure a user's heart rate and provide an alarm or similar notification when the heart rate exceeds a preset value, so that the user can regulate his or her behavior to maintain a desired heart rate. Some devices provide calming music, or similar soothing environmental sounds or white noise, to induce a user to undertake certain behaviors or cease undertaking certain behaviors responsible for the elevated heart rate.

However, no system exists that is specifically adapted to not only counteract a detected abnormal heart rate and related symptoms, but to treat the underlying condition responsible for the abnormal heart rate, eventually training a user's brain to compensate for the condition. To treat many of the described neurological and/or behavioral disorders, both the left and right sides of the brain must be engaged through a combination of sensations provided to a user responsive to the elevated heart rate.

A need exists for a system and method for treating a condition of a user that detects an abnormal heart rate and provides audible and/or tactile sensations to the user responsive to the abnormal heart rate.

A further need exist for a system and method that combines audible and tactile sensations, which are selectively or alternatingly provided to different areas of the user's body, to engage both sides of the user's brain.

A need also exists for a customizable and portable system and method, specifically adapted to treat conditions of a user, in which an abnormal heart rate is one symptom or serves as a secondary indicator for the underlying illness or disease.

The present embodiments meet these needs.

### SUMMARY

The present embodiments relate to a system for treating a condition of a user, when at least one of the symptoms of the condition includes an abnormal heart rate. An abnormal heart rate can include any detectable irregularity in the rate or rhythm of a user's pulse, including an elevated heart rate, a decreased heart rate, an irregular and/or erratic heart rate, or combinations thereof.

The system includes a heart rate monitor, in communication with the user. The heart rate monitor can be any type of measuring device able to accurately determine one or more heart rate parameters of the user. In an embodiment, the heart rate monitor can include one or more electrodes, appropriately positioned on the user's body, such as a R-T device with electrodes placed on the user's chest. In an alternate embodiment, the heart rate monitor could include an infrared device for measuring one or more parameters of the user's heart rate through detection of variations in emissivity, reflectivity, and/or opacity in the user's skin due to blood flow.

The heart rate monitor can be in direct or wireless communication with a processor, or similar controller, able to receive the measured heart rate parameters from the heart rate monitor and transmit signals to other system components. The processor can be in direct or wireless communication with one or more data storage media, within which at least one threshold value relating to the heart rate parameters is stored. Threshold values can be input into the data storage based on the user's age and/or the user's medical condition, can include customized values input by the user, a medical professional, or another third party, or the threshold values can include any number of selectable pre-recorded values.

The data storage can also include computer instructions for instructing the processor to compare the measured heart rate parameters to the one or more threshold values, and to transmit a signal when one or more heart rate parameters fall outside of one or more of the threshold values.

The system can also include an audio device, in direct or wireless communication with the processor, configured to provide audible sound to the user. For example, the audio device can include wireless earphones or earplugs, or a similar device having a speaker proximate to the user's ears. Responsive to the signal transmitted by the processor when one or more measured heart rate parameters exceed one or more threshold values, the audio device provides an audible sound to the user, that can be therapeutic and adapted to treat the user's condition, alleviate symptoms of the user's condition, or combinations thereof. In an embodiment, audible sounds can include alarm noise, a pre-recorded voice message, a synthesized voice-message, a voice message recorded by the user, a pre-recorded musical selection, a user-provided musical selection, a pre-recorded environmental sound or white noise, a user-recorded environmental sound, a sound patterned for therapeutic or restorative treatment, or combinations thereof.

The audio device can include one or more integrated or remote data storage media for storing sounds, music, and/or messages recorded by the user, a medical professional, or another third party, and for storing pre-recorded sounds, music, and/or messages. For example, the audio device can include a MP3 player, or similar device, having data storage within which the user or another individual can install downloaded musical selections. The audio device can also be configured to store and access recorded sounds stored within the one or more data storage media in communication with the processor. In an embodiment, the processor, the data storage, and the audio device can be integrated into a single, portable device.

The system can further include a tactile device in direct or wireless communication with the processor, configured to provide a tactile sensation, including therapeutic and/or rhythmic pulsations for treatment, to the user. In an embodiment, the tactile device can include one or more electrodes attached to the user's back. For example, the heart monitor and the tactile device can be integrated into a single wearable article, such as a band, strap, or belt, which can be wrapped around a user's torso to engage the user's chest with electrodes for measuring the user's heart rate parameters while engaging the user's back with electrodes for providing tactile sensations to the user.

In an embodiment, the audio device can be in communication with both the left and right ears of the user, for selectively or alternatingly providing audible sound to the left ear, then the right ear, in repetition, thereby simultaneously engaging both sides of the user's brain. Engagement of both the user's left brain and right brain facilitates treatment or alleviation of symptoms of various neurological and/or behavioral conditions, for treatment of the user.

Additionally, in an embodiment, the tactile device can be in communication with both the left and right sides of the user's body, such by adhering electrodes to the left and right sides of the user's back, arms, wrists, or hands. Tactile sensations can then be selectively or alternatingly provided to the left side of the body, then the right side, in repetition, for engaging both sides of the user's brain to treat and/or alleviate the symptoms of the user's condition.

The present embodiments also relate to a method for treating a condition of a user, the condition including an abnormal heart rate as a symptom. One or more heart rate parameters of the user are measured, then compared with at least one threshold value. Customized threshold values can be provided by the user or another individual, or preset threshold values can be used. Both an audible sound and a tactile sensation are provided to the user when one or more heart rate parameters exceed one or more of the threshold values. The audible sound, the tactile sensation, or combinations thereof, can be customized by the user, a medical professional, a therapist, or another individual, or the audible sound and/or the tactile sensation can be preselected.

The provision of audible sound can include selectively or alternatingly providing the sound to a user's left and right ears to engage both sides of the user's brain. Similarly, the provision of the tactile sensation can include selectively or alternatingly providing the sensation to left and right sides of the user's body to engage both sides of the brain.

The present systems and methods are thereby usable to treat and/or alleviate the symptoms of a wide variety of conditions, including behavioral disorders, anxiety, obsessive compulsive disorder, attention deficit disorder and attention deficit hyperactivity disorder, pervasive developmental disorders, post traumatic stress disorder, and other similar medical conditions and disorders.

### BRIEF DESCRIPTION OF THE DRAWINGS

In the detailed description of the embodiments presented below, reference is made to the accompanying drawings, in which:

FIG. 1 depicts a diagram of an embodiment of the present system.

FIG. 2A depicts a front view of an embodiment of the present system disposed on a user.

FIG. 2B depicts a back view of the embodiment of the system of FIG. 2A.

FIG. 3A depicts a front view of an embodiment of a heart rate monitor usable with the present system.

FIG. 3B depicts a top view of the heart rate monitor of FIG. 3A.

FIG. 3C depicts a back view of the heart rate monitor of FIG. 3A.

FIG. 4A depicts a front view of an embodiment of a processing unit and audio device usable with the present system.

FIG. 4B depicts an exploded side view of the unit of FIG. 4A.

FIG. 4C depicts a back view of the unit of FIG. 4A.

FIG. 5A depicts a front view of an embodiment of a heart rate monitor usable with the present system.

FIG. 5B depicts an exploded top view of the heart rate monitor of FIG. 5A.

FIG. 5C depicts a back view of the heart rate monitor of FIG. 5A.

The present embodiments are detailed below with reference to the listed Figures.

### DETAILED DESCRIPTION OF THE EMBODIMENTS

Before explaining the present embodiments in detail, it is to be understood that the embodiments are not limited to the particular descriptions and that the embodiments can be practiced or carried out in various ways.

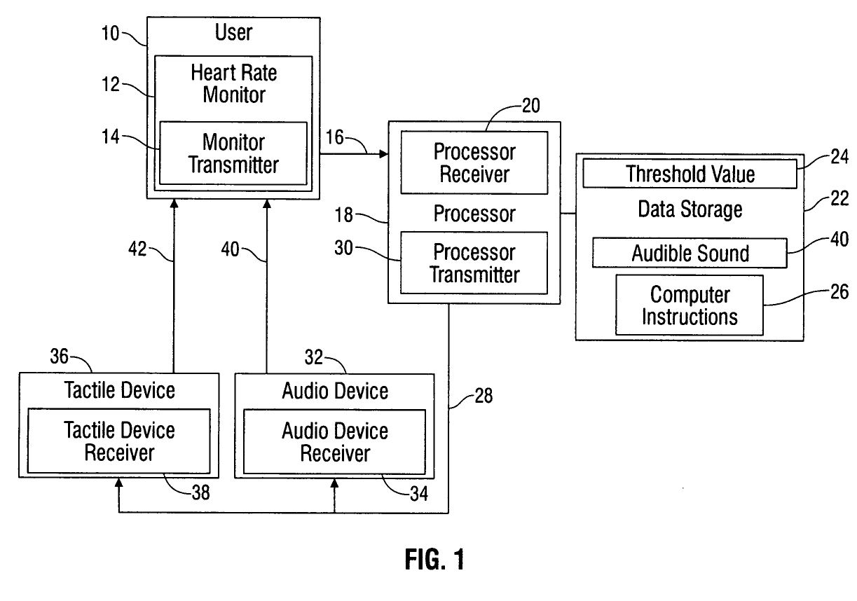

Referring now to FIG. 1, a diagram of an embodiment of the present system, usable to treat conditions of a user, is shown.

A user (10) is shown having a heart rate monitor (12) disposed thereon. The user (10) can include any individual, of any age or vocation, including adults and children, athletes, students, hospital patients, or any other individual who has been diagnosed or who may suffer from one or more heart conditions and/or behavioral or neurological disorders.

The heart rate monitor (12) can be any type of device able to measure at least one heart rate parameter of the user (10). Heart rate parameters can include the user's pulse rate, or other similar information that can be obtained through measurement of the user's heart using the heart rate monitor (12). In an embodiment, the heart rate monitor (12) can include one or more electrodes disposed on the user's body, such as a R-T device usable to obtain a partial or complete electrocardiograph. In another embodiment, the heart rate monitor (12) can include one or more infrared transmitters, paired with one or more infrared receivers, for measuring one or more heart rate parameters through detection of variations in emissivity, reflectivity, and/or opacity in the user's skin due to blood flow.

The heart rate monitor (12) is shown having a monitor transmitter (14), usable to transmit a heart rate parameter (16) to a processor (18). The processor (18) can include any type of controller or similar device able to receive, transmit, and process data, such as a computer, a cellular telephone, a MP3 player, or a similar device. In an embodiment, the processor (18) can be a portable device worn and/or carried by the user (10), in direct or wireless communication with the heart rate monitor (12). In an alternate embodiment, the processor (18) can be remote from the user (10) and in wireless communication with the heart rate monitor (12).

The processor (18) is depicted having a processor receiver (20), which can receive the heart rate parameter (16) transmitted by the heart rate monitor (12). The processor (18) is shown in communication with a data storage medium (22), which can include any number of integrated, removable, and/or remote data storage media usable to retain data accessible to the processor (18). In an embodiment, the data storage medium (22) can be integral with the processor (18). For example, the processor (18) and data storage medium (22) could both be part of a single device, such as a cellular telephone or portable music playback device, including a MP3 player. In an alternate embodiment, the data storage medium (22) can be remote from the processor (18) and in wireless communication with the processor (18).

The data storage medium (22) is shown containing a threshold value (24), related to the heart rate parameter (16). Any number of threshold values can be contained within the data storage medium (22). The threshold value (24) can include any value for which a triggered response is desired when the heart rate parameter (16) meets or exceeds the threshold value (24). In an embodiment, the threshold value (24) can be determined based on the age of the user (10), the condition or suspected condition of the user (10), one or more preset or pre-selected values, or any customized values input by the user (10), a medical professional, or any other third party.

Computer instructions (26) stored within the data storage medium (22) are usable to instruct the processor (18) to compare the heart rate parameter (16) to the threshold value (24), and to transmit a signal (28), if the heart rate parameter (16) falls outside of the threshold value (24). The processor (18) is shown having a processor transmitter (30), usable to transmit the signal (28).

The signal (28) is shown being transmitted to an audio device (32) having an audio device receiver (34) for receiving the signal (28). This signal can also be transmitted to a tactile device (36) having a tactile device receiver (38) for receiving the signal (28).

The audio device (32) can include any type of audio device capable of receiving the signal (28) and providing sound to the user (10). FIG. 1 depicts the data storage medium (22) containing an audible sound (40), which can be transmitted to the audio device (32) and provided by the audio device (32) to the user (10). For example, the audio device (32) can include wireless earphones and/or a MP3 player or similar music playback device.

While FIG. 1 depicts the audible sound (40) stored in the data storage medium (22), in an embodiment, the audio device (32) can include a separate integral, removable, and/or remote data storage medium for containing audible sounds, such as musical selections, alarm sounds, voice messages, therapeutic patterns or rhythms for treatment, or other similar sounds. In a further embodiment, the user (10), a medical professional, or another individual can select desired audible sounds to be provided to the user (10) for treatment of certain conditions, when the heart rate parameter (16) exceeds a specified threshold value (24). The audio device (32) could also include an input or selection means for enabling the user (10) to select various customized, user-provided, or pre-recorded audible sounds for provision under certain conditions. The audio device (32) could further include a microphone or similar recording device for enabling the user (10) or another individual to record customized voice messages or other audible sounds for playback, under certain selected conditions.

The tactile device (36) can include any type of device able to provide a tactile sensation, such as a therapeutic pulsation, to the user (10). Tactile devices can include vibrators, puslators, electrodes, heating elements, or similar therapeutic devices. In an embodiment, the tactile device (36) can include electrodes in communication with the user's body, which pulsate and/or vibrate in response to the signal (28) from the processor (18). For example, both the tactile device (36) and the heart rate monitor (12) could be part of a single integrated device, having a first set of electrodes on the user's chest for measuring the heart rate parameter (16), and a second set of electrodes on the user's back for providing a tactile sensation. FIG. 1 depicts the tactile device (36) providing a tactile sensation (42) to the user (10). The tactile sensation (42) can include vibrations, pulsations, pain, heat, cold, shock, or any other type of tactile sensation (42) able to influence the user (10), treat the user's condition, or alleviate the symptoms of the user's condition.

In an embodiment, the audible sound (40), the tactile sensation (42), or combinations thereof, can be coordinated to be selectively and/or alternatingly provided to the left and right sides of the user (10) for engaging both sides of the user's brain with therapeutic sounds and sensations to enhance the effective treatment of the user's condition. For example, the audio device (32) can include earphones or a similar device that simultaneously engages both the user's left and right ears, while the tactile device (36) can include pulsating and/or vibrating electrodes that engage the left and right sides of the user's lower back, or other parts the user's body, such as both arms. The audible sound (40) and/or the tactile sensation (42) can be provided first to the user's left side, then to the user's right side, then alternating in repetition until the heart rate parameter (16) no longer exceeds the threshold value (24).

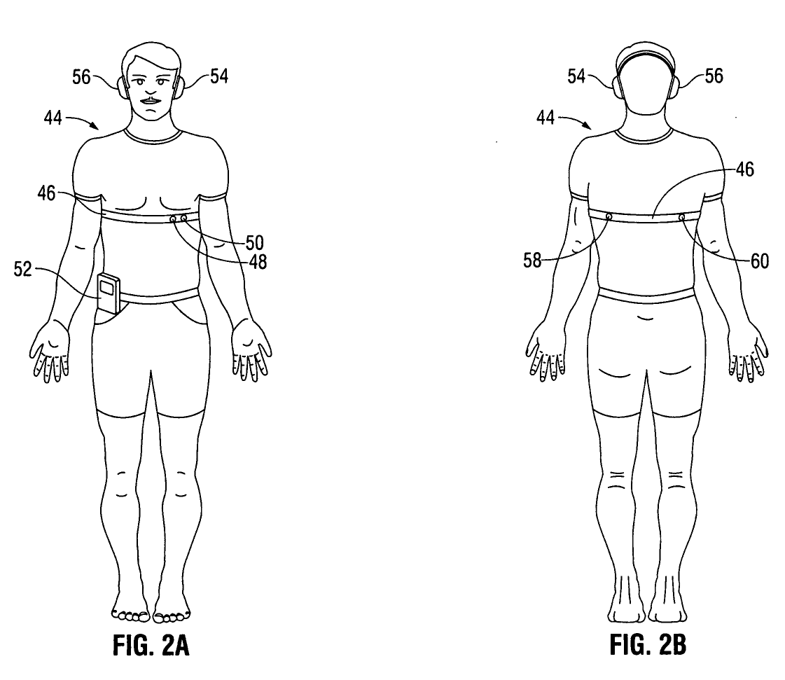

FIG. 2A depicts a front view of an embodiment of the present system, disposed about the body of a user (44). The depicted embodiment includes a single band (46), which can be an adjustable or elastic band, belt, strap, or similar article able to be fitted closely against the user's chest and back. The band (46) includes a heart rate monitor, which has a first electrode (48) and a second electrode (50) disposed against the user's chest on either side of the approximate location of the user's heart.

While FIG. 2A depicts two electrodes (48, 50), it should be understood that additional electrodes could be utilized. For example, the band (46) could be made sufficiently broad to include from two to six electrodes, positioned at appropriate lead locations on the user's chest and left side for acquiring a partial or complete electrocardiograph. Additionally, while FIG. 2A depicts two electrodes (48, 50) within a single band (46) secured around the user's torso, it should be understood that other arrangements of electrodes able to measure heart rate parameters of the user (44) can also be utilized. For example, separate bands containing electrodes could be wrapped around a user's wrists, ankles, thighs, and/or shoulders, or electrodes could be directly adhered to the user's chest independent of a band or strap. Any arrangement of electrodes utilized can be in wired or wireless communication with one or more processors adapted to receive data from the electrodes.

FIG. 2A depicts a portable processing unit (52) worn about the user's waist. The depicted processing unit (52) can be in wired or wireless communication with the electrodes (48, 50), can include a processor adapted for receiving parameters from the electrodes (48, 50), and data storage containing computer instructions for instructing the processor to compare parameters from the electrodes (48, 50) to one or more threshold values in the data storage. In an embodiment, the processing unit (52) can be in wireless communication with one or more remote data storage media.

The processing unit (52) can also include an audio device, such as a portable music player. Songs or other audible sounds, such as voice messages, can be stored in the data storage medium of the processing unit (52). The user (44) is shown wearing a first portable ear piece (54) over the left ear and a second portable ear piece (56) over the right ear. The portable ear pieces (54, 56) can be in direct or wireless communication with the processing unit (52) for providing audible sound selectively and/or alternatingly to the user's left and right ears responsive to parameters received from the electrodes (48, 50) and processed by the processing unit (52). For example, in response to an elevated heart rate detected by the electrodes (48, 50), the processing unit (52) can cause a first audible sound to be provided to the ear pieces (54, 56) simultaneously, while in response to a severely elevated heart rate, the processing unit (52) can cause a different audible sound to be provided alternatingly to the first and second ear pieces (54, 56) to engage both sides of the user's brain.

FIG. 2B depicts a rear view of the embodiment of the system shown in FIG. 2A. The user (44) is shown with the band (46) secured about the torso and the first and second ear pieces (54, 56) secured over the left and right ears, respectively.

The band (46) is shown having a first tactile electrode (58) positioned over the left side of the user's back and a second tactile electrode (60) positioned over the right side of the user's back. The tactile electrodes (58, 60) can be in wired or wireless communication with the processing unit (52) and can provide a tactile sensation selectively and/or alternatingly to the right and left portions of the user's back responsive to parameters received by the processing unit (52) from the heart monitor electrodes (depicted in FIG. 2A).

While FIGS. 2A and 2B depict two heart monitoring electrodes (48, 50) and two tactile electrodes (58, 60) contained within a single band (46) engaging a user's torso, it should be understood that other arrangements are also usable without departing from the scope of the invention. For example, separate heart monitoring and tactile response articles or electrodes can be secured to the user (44). Additionally, the position and number of heart monitor electrodes and tactile electrodes can be varied.

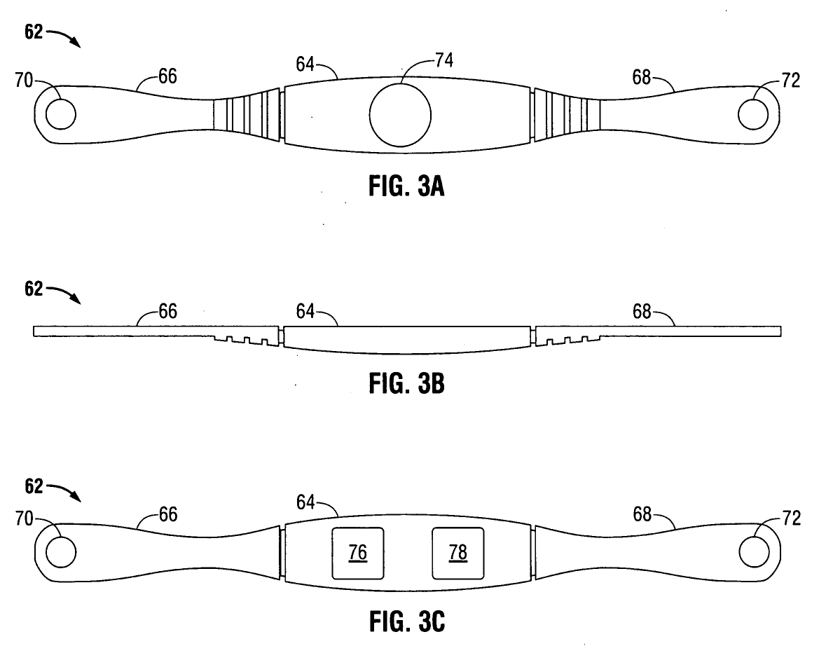

Referring now to FIGS. 3A, 3B, and 3C, an embodiment of a heart rate monitor (62) usable with the present system is shown. The heart rate monitor (62) is depicted as a band having a central section (64), with a first side section (66) and a second side section (68) disposed on opposing ends. The length of the band can be sufficient to secure around a user's torso. In an embodiment, the band can be sized to be secured about a user's wrist or ankle for measuring the user's pulse rate at that location. In an embodiment, the side sections (66, 68) of the band can include buckles, snaps, adhesive, or other hook-and-loop or similar fasteners for enabling adjustment to fit users of varying size.

FIGS. 3A and 3C depict the first and second side sections (66, 68) containing a first securing member (70) and a second securing member (72), respectively. The securing members (70, 72) can be snaps, buttons, or similar members configured to engage each other to secure around a user, or the securing members can include adhesive or similar means for engaging directly to a user's body or to an article of clothing worn by the user. In an embodiment, the securing members (70, 72) or another portion of the side sections (66, 68) can include tactile electrodes for engagement with the user's back, as described previously.

The central section (64) is shown containing a battery chamber (74) on its front side, in which a power source can be positioned for powering electrodes (76, 78) disposed on the back side of the central section (64). The electrodes (76, 78) can be positioned along the band such that when the band is secured around a user, the electrodes (76, 78) are properly positioned to measure the user's heart rate. The electrodes (76, 78) can be in wired or wireless communication with a processor for comparing the measured heart rate parameters of the user with threshold values.

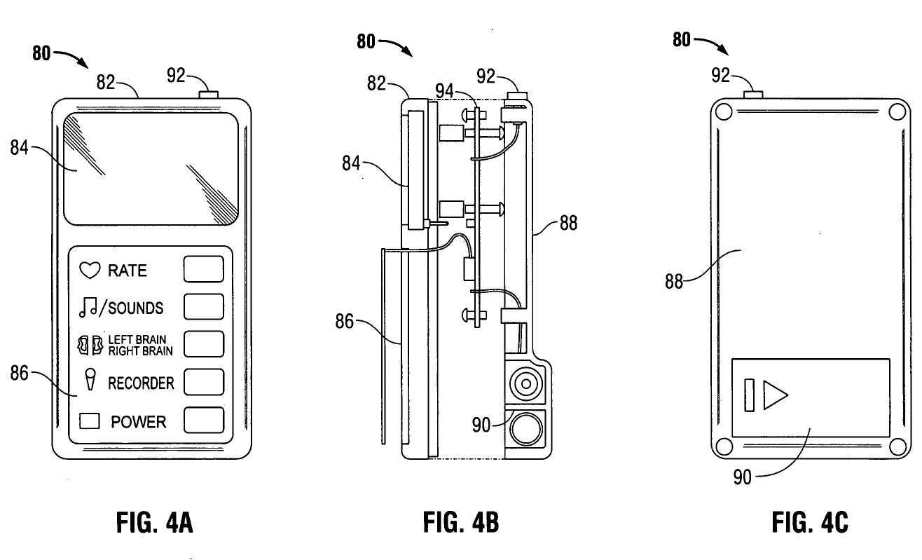

Referring now to FIGS. 4A, 4B, and 4C, an embodiment of a processing unit (80) usable with the present system is shown. The processing unit (80) can include a processor for receiving heart rate parameters from electrodes and transmitting audible sounds and tactile sensations to respective output devices, data storage for containing processing software, threshold heart rate parameters, and recorded music and sound files, a portable power source, and related displays, input devices, and interfaces.

FIG. 4A depicts a front view of the processing unit (80), which includes a front housing member (82), an upper display (84), and a lower display (86). The displays (84, 86) can include any type of visual display or graphical user interface, such as a liquid crystal display. The displays (84, 86) can further include tactile response and/or touch-screen technology, or similar input members, such as buttons or switches, or voice-recognition technology. FIG. 4A depicts the upper display (84) as a general display, while the lower display (86) includes a depiction of the user's heart rate; the presence, volume, or an identification of an audible sound; the stimulation of the user's brain undertaken by the processing unit (80); a recording indicator; and a power indicator. Other display arrangements and indicators can also be utilized. An audio jack (92) is also visible, which can be used for engagement with an audio device, such as a headphones. In an embodiment, the processing unit (80) can be in wireless communication with headphones, or a similar output device, and the audio jack (92) can be omitted.

FIG. 4C depicts a back view of the processing unit (80), which includes a back housing member (88) having a battery chamber (90) disposed therein for containing a power source. FIG. 4C also depicts the audio jack (92), however it should be noted that the audio jack (92) can be omitted in some embodiments, such as when wireless headphones or a similar wireless device is used to provide sound to a user.

FIG. 4B depicts an exploded side view of the processing unit (80), which displays a circuit board (94) disposed between the front and back housing sections (82, 88). The back housing section (88) is shown having the audio jack (92) disposed thereon. The circuit board (94) can include a processor and associated data storage, which can contain computer instructions for instructing the processor to receive heart rate parameters from heart rate monitor electrodes, compare the parameters to threshold values in the data storage, and cause audio and/or tactile output responsive to the results of the comparison. The processor and data storage can also manipulate information on the displays (84, 86). The depicted processing unit (80) can also be provided with other functionality, such as the ability to function as a conventional audio recording, storage, and playback device, a portable television or similar display device, a portable computer, a cellular telephone, and other similar devices.

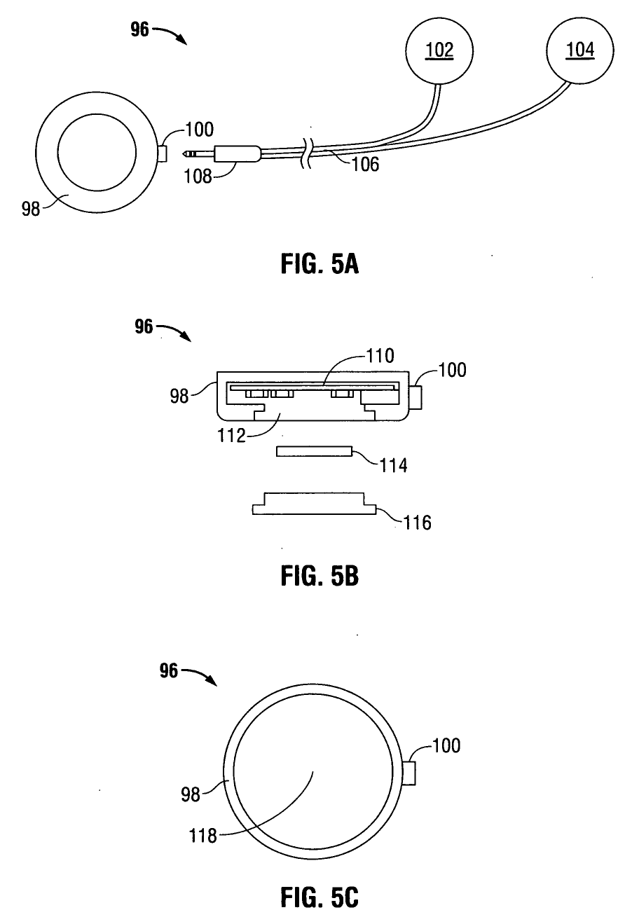

Referring now to FIGS. 5A, 5B, and 5C, an alternate embodiment of a heart rate monitor (96) usable with the present system is shown. The heart rate monitor (96) has a housing (98), which can be made from molded silicon or a similar material able to comfortably adhere to a user's body or article of clothing. The heart rate monitor has an output jack (100), usable for wired communication with a first electrode (102) and a second electrode (104), which can be usable to measure heart rate, to provide tactile sensations, or combinations thereof. The housing (98) can also contain one or more electrodes for measuring a heart rate parameter or providing a tactile sensation at the location where the housing is positioned on the user's body. While FIG. 5A depicts the electrodes (102, 104) configured for wired communication with the heart rate monitor (96) via a dual wire (106) attached to a plug (108) for insertion into the output jack (100), in an embodiment, the electrodes (102, 104) and the heart rate monitor (96) can be in wireless communication. Additionally, while FIG. 5A depicts the heart rate monitor (96) having a single output jack (100), the heart rate monitor (96) could have multiple output jacks for simultaneous communication with heart rate electrodes, vibrating electrodes, sound devices, or other output devices. Alternatively, the heart rate monitor (96) could be in wireless communication with one or more electrodes.

FIG. 5B depicts an exploded top view of the heart rate monitor (96), having the housing (98) and output jack (100), disposed over a circuit board (110). The depicted heart rate monitor (96) is shown having a battery chamber (112), adapted to contain a coin cell battery (114), and a complementary battery cover (116).

FIG. 5C depicts a back view of the heart rate monitor (96), in which the output jack (100) is visible. The back side of the housing (98) is shown having an adhesive backing (118), which can be used to secure the heart rate monitor (96) to a user. Other attachment means can also be used, such as hooks, Velcro™ or other hook and loop fasteners, snaps, clasps, pins, buckles, or other similar fastening members.

While these embodiments have been described with emphasis on the embodiments, it should be understood that within the scope of the appended claims, the embodiments might be practiced other than as specifically described herein.

What is claimed is:

1. A system for facilitating treatment of a condition of a user, wherein a symptom of the condition comprises an abnormal heart rate, the system comprising:
a heart rate monitor in communication with the user, wherein the heart rate monitor measures at least one heart rate parameter of the user;
a processor in communication with the heart rate monitor and at least one data storage medium, wherein the processor receives said at least one heart rate parameter from the heart rate monitor, and wherein said at least one data storage media comprises at least one threshold value relating to said at least one heart rate parameter and computer instructions for instructing the processor to compare said at least one heart rate parameter to said at least one threshold value and to transmit a signal when said at least one heart rate parameter meets or exceeds said at least one threshold value;
an audio device in communication with the processor and the user, wherein the audio device receives the transmitted signal from the processor and provides an audible sound to the user responsive to the transmitted signal, and wherein the audible sound is adapted to treat the condition of the user, alleviate symptoms of the condition, or combinations thereof; and
a tactile device in communication with the processor and the user, wherein the tactile device receives the transmitted signal from the processor and provides a tactile sensation to the user responsive to the transmitted signal, and wherein the tactile sensation is adapted to treat the condition of the user, alleviate symptoms of the condition, or combinations thereof.

2. The system of claim 1, wherein the heart rate monitor, the processor, said at least one data storage medium, the audio device, the tactile device, or combinations thereof, are in wireless communication.

3. The system of claim 1, wherein the heart rate monitor comprises at least one electrode, at least one infrared device, or combinations thereof, for measuring said at least one heart rate parameter, and wherein the heart rate monitor further comprises a transmitter for transmitting said at least one heart rate parameter to the processor.

4. The system of claim 1, wherein the audio device is in communication with a left ear of the user and a right ear of the user, and wherein the audio device selectively or alternatingly provides the audible sound to the left ear, the right ear, or combinations thereof, for engaging both a left side of the user's brain and a right side of the user's brain to treat the condition of the user, alleviate symptoms of the condition, or combinations thereof.

5. The system of claim 4, wherein the audio device is configured to provide the audible sound to the left ear, then provide the audible sound to the right ear, then continue providing the audible sound to the left ear, then the right ear, in repetition, until said at least one heart rate parameter no longer exceeds said at least one threshold value.

6. The system of claim 1, wherein the tactile device is in communication with a left side of the user's body and a right side of the user's body, and wherein the tactile device selectively or alternatingly provides the tactile sensation to the left side of the user's body, the right side of the user's body, or combinations thereof, for engaging both a left side of the user's brain and a right side of the user's brain to treat the condition of the user, alleviate symptoms of the condition, or combinations thereof.

7. The system of claim 6 wherein the tactile device is configured to provide the tactile sensation to the left side of the user's body, then provide the tactile sensation to the right side of the user's body, then continue providing the tactile sensation to the left side, then the right side, in repetition, until said at least one heart rate parameter no longer exceeds said at least one threshold value.

8. The system of claim 1, wherein the abnormal heart rate comprises an elevated heart rate, a decreased heart rate, an irregular heart rate, or combinations thereof.

9. The system of claim 1, wherein the audible sound comprises an alarm noise, a pre-recorded voice message, a synthesized voice message, a user-recorded voice message, a pre-recorded musical selection, a user-recorded musical selection, a pre-recorded environmental sound, a user-recorded environmental sound, or combinations thereof.

10. The system of claim 1, wherein the tactile sensation comprises pulsations, vibrations, heat, cold, pain, pressure, or combinations thereof.

11. The system of claim 1, wherein the condition of the user a behavioral disorder, a pervasive developmental disorder, anxiety, obsessive-compulsive disorder, post traumatic stress disorder, or combinations thereof.

12. The system of claim 1, wherein said at least one threshold value is determined using the condition of the user, an age of the user, a pre-selected value, a user-selected value, or combinations thereof.

13. The system of claim 1, wherein the tactile devices comprises at least one electrode in communication with a back of the user.

14. A method for facilitating treatment of a condition of a user, wherein a symptom of the condition comprises an abnormal heart rate, the method comprising the steps of:
measuring at least one heart rate parameter of the user;
comparing said at least one heart rate parameter to at least one threshold value;
providing an audible sound to the user when said at least one heart rate parameter exceeds said at least one threshold value, wherein the audible sound is adapted to treat the condition of the user, alleviate symptoms of the condition, or combinations thereof; and
providing a tactile sensation to the user when said at least one heart rate parameter exceeds said at least one threshold value, wherein the tactile sensation is adapted to treat the condition of the user, alleviate symptoms of the condition, or combinations thereof.

15. The method of claim 14, wherein the step of providing the audible sound to the user comprises selectively or alternatingly providing the audible sound to a left ear of the user, a right ear of the user, or combinations thereof, for engaging both a left side of the user's brain and a right side of the user's brain to treat the condition of the user, alleviate symptoms of the condition, or combinations thereof.

16. The method of claim 14, wherein the step of providing the tactile sensation to the user comprises selectively or alternatingly providing the audible sound to a left side of the user's body, a right side of the user's body, or combinations thereof, for engaging both a left side of the user's brain and a right side of the user's brain to treat the condition of the user, alleviate symptoms of the condition, or combinations thereof.

17. The method of claim 14, further comprising the step of providing a user customized threshold value for comparison with said at least one measured heart rate parameter of the user.

18. The method of claim 14, further comprising the step of providing a customized audible sound, a customized tactile sensation, or combinations thereof, for provision to the user.

19. The method of claim 14, wherein the step of providing the audible sound to the user comprises providing an alarm noise, a pre-recorded voice message, a synthesized voice message, a user-recorded voice message, a pre-recorded musical selection, a user-recorded musical selection, a pre-recorded environmental sound, a user-recorded environmental sound, or combinations thereof, to the user.

20. The method of claim 14, wherein the step of providing the tactile sensation to the user comprises providing pulsations, vibrations, heat, cold, pain, pressure, or combinations thereof, to the user.
